(19) United States
(12) Patent Application Publication
(10) Pub. No.: US 2026/0183441 A1
(43) Pub. Date: Jul. 2, 2026

(54) Bottle Cap Soaking Bay

(71) Applicant: Michael D DeLeeuw, Washington, UT (US)

(72) Inventor: Michael D DeLeeuw, Washington, UT (US)

(21) Appl. No.: 19/435,140

(22) Filed: Dec. 29, 2025

Related U.S. Application Data
(60) Provisional application No. 63/739,390, filed on Dec. 27, 2024.

(51) Int. Cl. A61L 9/04; A44C 9/00; A44C 15/00

(52) U.S. Cl. CPC A61L 9/04; A44C 9/0053; A44C 15/002; A61L 2209/13

(57) ABSTRACT

The bottle cap soaking bay has top cap and a bottom cap. The bottom cap has a body, a bay, threads, ridges and a safety seal. The bottom cap had a tapered cylindrical shape. The bay is a watertight container for storing an oil-infused therapeutic device. The threads allow the top cap to be secured to the top of the body, sealing the bay, where the therapeutic device may soak in oil over time.

### FIELD OF THE INVENTION

The present invention relates generally to containers for liquids. The present invention relates more specifically to oil containers for refreshing oil-infused aromatherapy devices.

### BACKGROUND OF THE INVENTION

Aromatherapy provides beneficial effects through positive sensory feedback that humans may experience when exposed to certain fragrances and scents. Essentials oils provide many beneficial effects when used in aromatherapy. A wearable oil-infused therapeutic device provides aromatherapy that moves with the user. The wearable oil-infused therapeutic device needs to be soaked in oil to replace the oil that evaporates during use.

It is an objective of the present invention to provide a convenient container to store and soak a wearable oil-infused therapeutic device. It is a further objective of the present invention to provide a container that replaces the cap of a bottle of oil, providing a convenient solution to store the therapeutic device on top of the bottle of oil. It is a further objective to provide an all-in-one system for transporting a therapeutic device and oil.

### BRIEF DESCRIPTION OF THE DRAWINGS

FIG. 1 is a top front right perspective view of the present invention.

FIG. 2 is a bottom rear left perspective view of the present invention.

FIG. 3 is a front elevation view of the present invention.

FIG. 4 is a top plan view of the present invention.

FIG. 5 is an exploded top front right perspective view of the present invention, showing the top cap removed from the bottom cap.

FIG. 6 is an exploded top front right perspective view of the present invention, showing the top cap removed and a therapeutic device stored within the bay.

FIG. 7 is an exploded bottom front right perspective view of the present invention, showing the top cap removed.

FIG. 8 is an exploded front view of the present invention, without the safety seal and showing the top cap removed.

FIG. 9 is a cross-sectional view of the present invention, without the safety seal and showing the top cap removed.

### DETAILED DESCRIPTION OF THE INVENTION

All illustrations of the drawings are for the purpose of describing selected versions of the present invention and are not intended to limit the scope of the present invention.

The present invention may be used to replace the original cap of a bottle of oil, providing a convenient storage location for the present invention. The present invention may be used to soak a wearable oil-infused aromatherapy device in oil while conveniently storing the device on top of the bottle of oil.

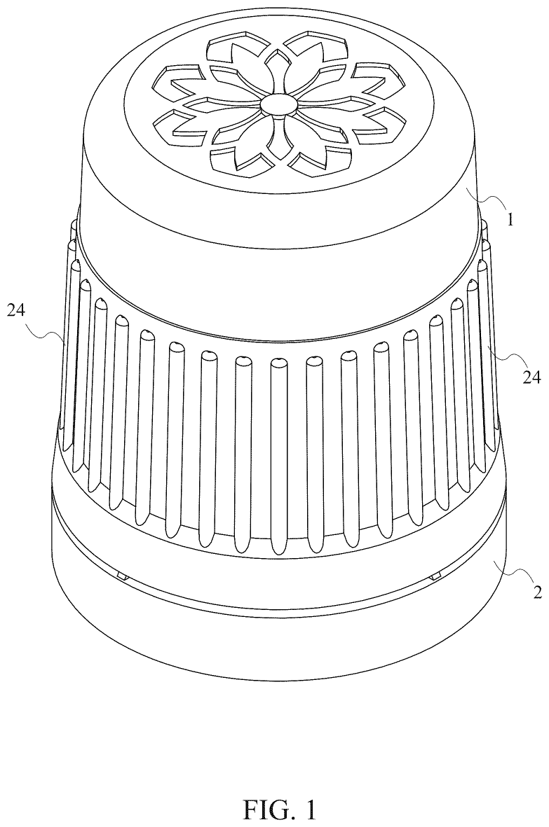

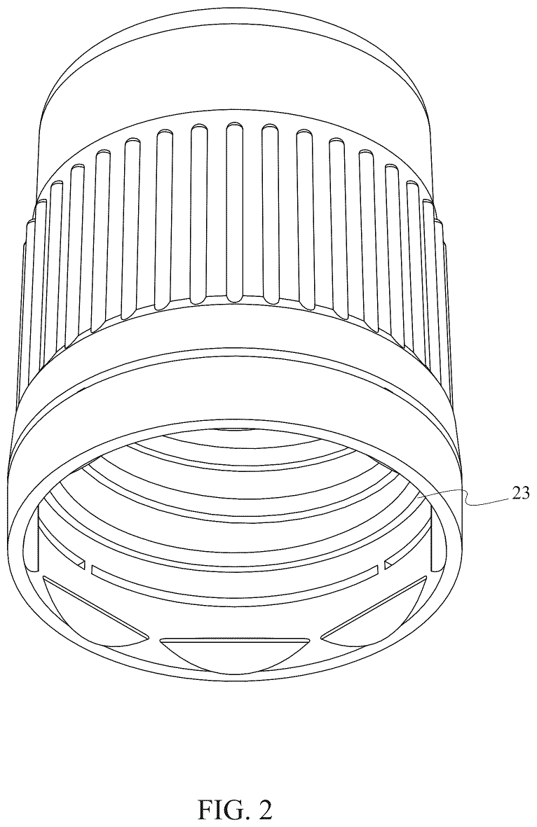

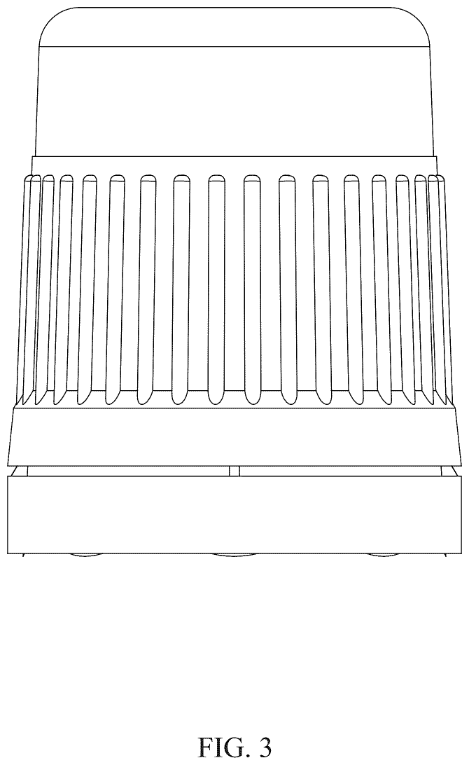

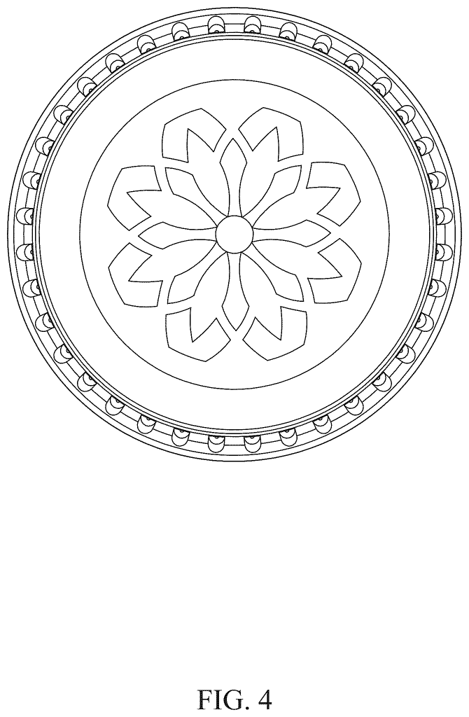

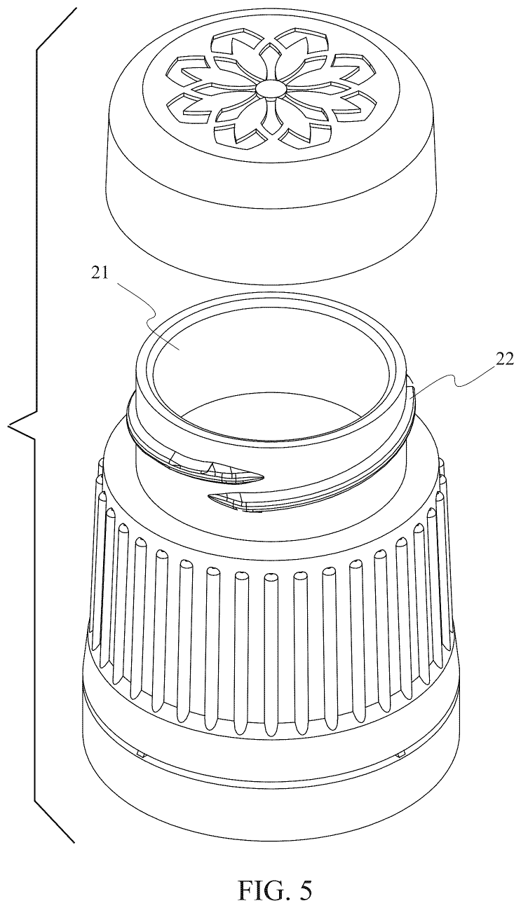

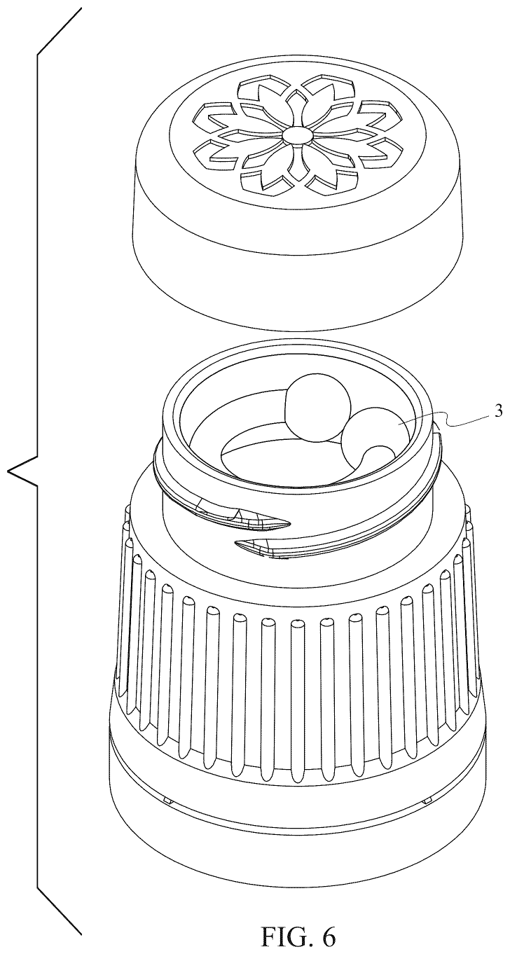

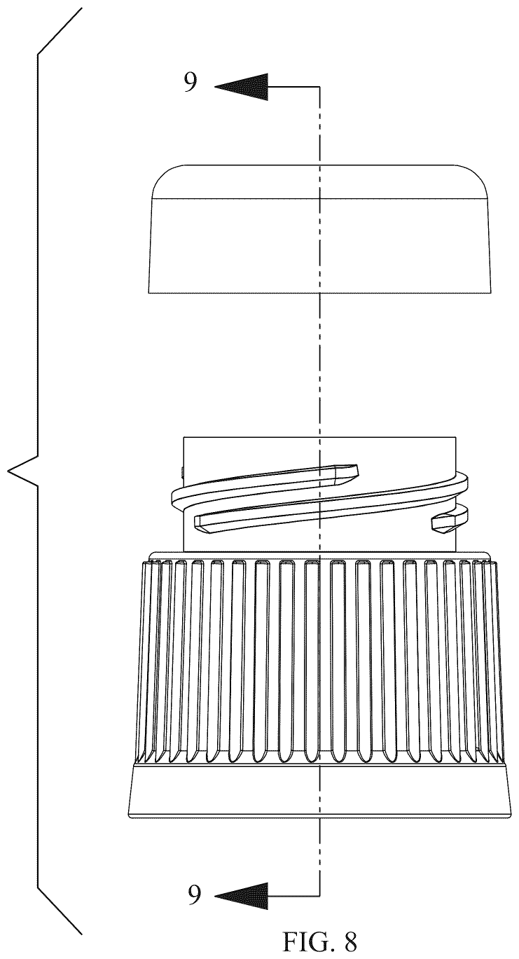

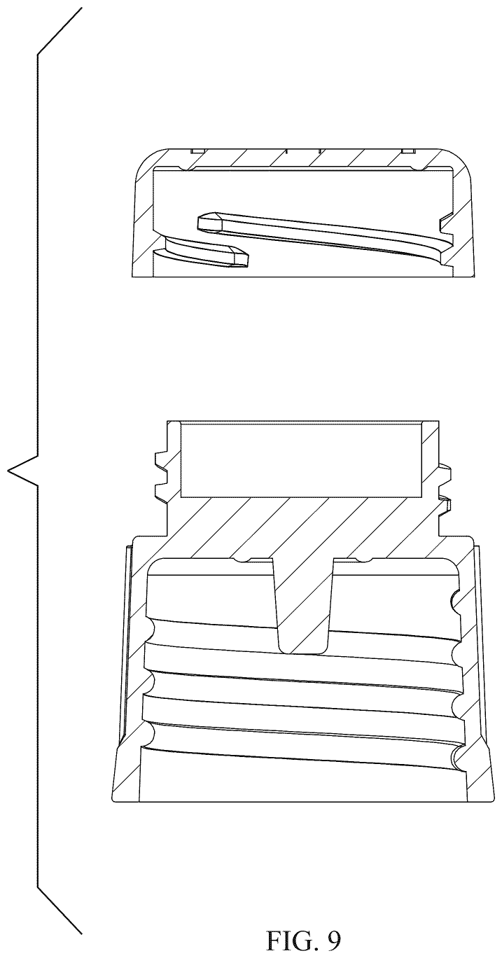

Referring to FIGS. 1-9, the present invention represents a bottle cap soaking bay 21. The bottle cap comprises a top cap 1, a bottom cap 2 and an essence ring 3. The top cap 1 comprises a threaded inner surface 11. The bottom cap 2 comprises a body, a bay 21, a threaded outer surface 22, a bottle threaded surface 23, a plurality of ridges 24 and a safety seal. The top cap 1 mechanically couples to the bottom cap 2. The essence ring 3 is positioned within the bottom cap 2. As a result, the top cap 1 and bottom cap 2 are sealed around the essence ring 3. The bottom cap 2 has a tapered cylindrical shape. The bay 21 is a sealed area when the top cap 1 is engaged to the bottom cap 2. The bay 21 houses the essence ring 3. The bay 21 is a watertight container for storing an oil-infused therapeutic device. For example, the bay 21 may store an oil-infused elastomeric therapeutic device. The threaded outer surface 22 is positioned along the top side of the bottom cap 2. Consequently, the threaded outer surface 22 enables the top cap 1 to be secured to the top of the bottom cap 2, sealing the bay 21. The threaded outer surface 22 engages with the top cap 1. Accordingly, the top cap 1 may be rotatably fastened to the bottom cap 2. The plurality of ridges 24 is integrated along the outer surface of the bottom cap 2. The plurality of ridges 24 is positioned below the threaded outer surface 22. Thus, the plurality of ridges 24 aid the user's grip when turning the bottom cap 2. The bottle threaded surface 23 integrates along the bottom of the bottom cap 2. The bottle threaded surface 23 integrates along the inner surface of the bottom cap 2. So, the bottle threaded surface 23 enables the bottom cap 2 to be secured to the top of a bottle. The safety seal is connected to the bottom of the body. When the bottom cap 2 is placed on a bottle, the safety seal creates an airtight seal on the bottle. Further, the bottle threaded surface 23 engages to an external bottle.

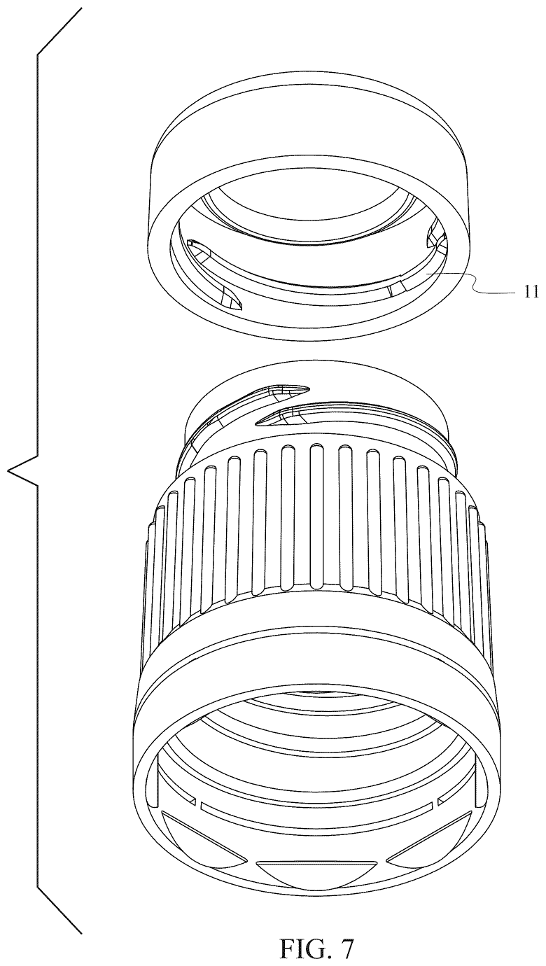

In reference to FIG. 7, the threaded inner surface 11 integrates along the inner surface of the top cap 1. The threaded inner surface 11 engages with the threaded outer surface 22 of the bottom cap 2. As a result, the top cap 1 may secure to the bottom cap 2. The top cap 1 is rotatably tightened to the bottom cap 2 to seal the bay 21. Consequently, the present invention may be stored on top of a bottle of oil. The bottle cap soaking bay 21 is removed from the bottle of oil, and the top cap 1 is removed from the bottom cap 2. The bay 21 may be filled with 2-3 drops of oil, and the therapeutic device is placed within the bay 21. Over time the oil will be absorbed into the therapeutic device. For example, the therapeutic device may be soaked for 4-6 hours or overnight. The top cap 1 is replaced on the bottom cap 2, covering and sealing the bay 21. The bottom cap 2 is then screwed onto the top of the bottle of oil. Once the therapeutic device has soaked for the desired duration, the top cap 1 is removed to open the bay 21, and the therapeutic device is removed from the bay 21 and placed on the user's body.

Although the invention has been explained in relation to its preferred embodiment, it is to be understood that many other possible modifications and variations can be made without departing from the spirit and scope of the invention as hereinafter claimed.

What is claimed is:

1. A bottle cap soaking bay comprising:
a top cap;
a bottom cap;
an essence ring;
the top cap comprising a threaded inner surface;
the bottom cap comprising a bay, a threaded outer surface, a bottle threaded surface, and a plurality of ridges;
the top cap mechanically coupling to the bottom cap; and
the essence ring being positioned within the bottom cap.

2. The bottle cap soaking bay as claimed in claim 1 wherein the bottom cap having a tapered cylindrical shape.

3. The bottle cap soaking bay as claimed in claim 1 comprising:
the bay being a sealed area when the top cap is engaged to the bottom cap; and
the bay housing the essence ring.

4. The bottle cap soaking bay as claimed in claim 1 comprising:
the threaded outer surface being positioned along the top side of the bottom cap; and
the threaded outer surface engaging with the top cap.

5. The bottle cap soaking bay as claimed in claim 1 comprising:
the plurality of ridges being integrated along the outer surface of the bottom cap; and
the plurality of ridges being positioned below the threaded outer surface.

6. The bottle cap soaking bay as claimed in claim 1 comprising:
the bottle threaded surface integrating along the bottom of the bottom cap; and
the bottle threaded surface integrating along the inner surface of the bottom cap.

7. The bottle cap soaking bay as claimed in claim 6 wherein the bottle threaded surface engaging an external bottle.

8. The bottle cap soaking bay as claimed in claim 1 comprising:
the threaded inner surface integrating along the inner surface of the top cap; and
the threaded inner surface engaging with the threaded outer surface of the bottom cap.

9. The bottle cap soaking bay as claimed in claim 8 wherein the top cap being rotatably tightened to the bottom cap to seal the bay.

10. A bottle cap soaking bay comprising:
a top cap;
a bottom cap;
an essence ring;
the top cap comprising a threaded inner surface;
the bottom cap comprising a bay, a threaded outer surface, a bottle threaded surface, and a plurality of ridges;
the top cap mechanically coupling to the bottom cap;
the essence ring being positioned within the bottom cap;
the bottom cap having a tapered cylindrical shape; and
the bay being a sealed area when the top cap is engaged to the bottom cap.

11. The bottle cap soaking bay as claimed in claim 10 wherein the bay housing the essence ring.

12. The bottle cap soaking bay as claimed in claim 10 comprising:
the threaded outer surface being positioned along the top side of the bottom cap;
the threaded outer surface engaging with the top cap;
the plurality of ridges being integrated along the outer surface of the bottom cap; and
the plurality of ridges being positioned below the threaded outer surface.

13. The bottle cap soaking bay as claimed in claim 10 comprising:
the bottle threaded surface integrating along the bottom of the bottom cap; and
the bottle threaded surface integrating along the inner surface of the bottom cap.

14. The bottle cap soaking bay as claimed in claim 13 wherein the bottle threaded surface engaging an external bottle.

15. The bottle cap soaking bay as claimed in claim 10 comprising:
the threaded inner surface integrating along the inner surface of the top cap; and
the threaded inner surface engaging with the threaded outer surface of the bottom cap.

16. The bottle cap soaking bay as claimed in claim 15 wherein the top cap being rotatably tightened to the bottom cap to seal the bay.

17. A bottle cap soaking bay comprising:
a top cap;
a bottom cap;
an essence ring;
the top cap comprising a threaded inner surface;
the bottom cap comprising a bay, a threaded outer surface, a bottle threaded surface, and a plurality of ridges;
the top cap mechanically coupling to the bottom cap;
the essence ring being positioned within the bottom cap;
the bottom cap having a tapered cylindrical shape;
the bay being a sealed area when the top cap is engaged to the bottom cap;
the bay housing the essence ring;
the threaded outer surface being positioned along the top side of the bottom cap; and
the threaded outer surface engaging with the top cap.

18. The bottle cap soaking bay as claimed in claim 17 comprising:
the plurality of ridges being integrated along the outer surface of the bottom cap;
the plurality of ridges being positioned below the threaded outer surface;
the bottle threaded surface integrating along the bottom of the bottom cap; and
the bottle threaded surface integrating along the inner surface of the bottom cap.

19. The bottle cap soaking bay as claimed in claim 18 wherein the bottle threaded surface engaging an external bottle.

20. The bottle cap soaking bay as claimed in claim 17 comprising:
the threaded inner surface integrating along the inner surface of the top cap;
the threaded inner surface engaging with the threaded outer surface of the bottom cap; and
the top cap being rotatably tightened to the bottom cap to seal the bay.
